(19) United States
(12) Patent Application Publication
(10) Pub. No.: US 2013/0172686 A1
(43) Pub. Date: Jul. 4, 2013

(54) SYSTEMS AND METHODS FOR DETERMINING PHYSIOLOGICAL INFORMATION USING AUTOCORRELATION WITH GAPS

(71) Applicants: Paul S. Addison, Edinburgh (GB); James Dripps, West Linton (GB); James Ochs, Seattle (GB); James Watson, Dunfermline (GB)

(72) Inventors: Paul S. Addison, Edinburgh (GB); James Dripps, West Linton (GB); James Ochs, Seattle (GB); James Watson, Dunfermline (GB)

(73) Assignee: Nellcor Puritan Bennett Ireland, Mervue (IE)

(21) Appl. No.: 13/343,421

(22) Filed: Jan. 4, 2012

(51) Int. Cl. A61B 5/00

(52) U.S. Cl. 600/300

(57) ABSTRACT

A patient monitoring system may receive a physiological signal having gap portions in the received data. The gap portions may be identified and a plurality of morphology metric signals may be modified based on the identified gap portions. The morphology metric signals may be modified based on the identified gaps, and a combined autocorrelation sequence may be generated based on the modified morphology metric signals. The combined autocorrelation sequence may be used to determine physiological information.

The present disclosure relates to physiological signal processing, and more particularly relates to determining physiological information such as respiration information from a physiological signal having gaps of unusable information using autocorrelation.

### SUMMARY

A patient monitoring system may be configured to determine physiological information such as respiration information from a physiological signal such as a photoplethysmograph (PPG) signal. For example, a PPG signal may exhibit amplitude and frequency modulation based on the respiration of a patient. A plurality of morphology metric signals may be generated from the physiological signal. A combined autocorrelation sequence may be generated from the plurality of morphology metric signals, and the combined autocorrelation sequence may be used to determine the physiological information.

In some instances, a physiological signal such as a PPG signal may include one or more gaps of undesirable data due to motion artifacts, arrhythmia, measurement error, or other reasons. This may reduce the amount of data available to determine the physiological information. The gaps may be identified and the determination of the autocorrelation sequence may be modified to reduce or ignore the contribution of the gap portions while retaining the remainder of the information for purposes of generating the combined autocorrelation sequence.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with some embodiments of the present disclosure;

FIG. 2 is a block diagram of the illustrative patient monitoring system of FIG. 1 coupled to a patient in accordance with some embodiments of the present disclosure;

FIG. 3 shows a block diagram of an illustrative signal processing system in accordance with some embodiments of the present disclosure;

FIG. 4 shows an illustrative PPG signal, a first derivative of the PPG signal, and a second derivative of the PPG signal in accordance with some embodiments of the present disclosure;

FIG. 5 is a flow diagram showing illustrative steps for determining respiration information from a photoplethysmograph signal in accordance with some embodiments of the present disclosure;

FIG. 6 depicts a plot of a physiological signal having a single gap portion and an exemplary modified morphology metric signal generated from the physiological signal;

FIG. 7 depicts exemplary autocorrelation plots generated based on the physiological signal of FIG. 6;

FIG. 8 depicts a plot of a physiological signal having two gap portions and an exemplary modified morphology metric signal generated from the physiological signal;

FIG. 9 depicts exemplary autocorrelation plots generated based on the physiological signal of FIG. 8; and

FIG. 10 depicts a plot of a modified morphology metric signal and exemplary autocorrelation plots based on the modified morphology metric signal.

### DETAILED DESCRIPTION OF THE FIGURES

The present disclosure is directed towards determining physiological information from a physiological signal having one or more gaps of undesirable data due to motion artifacts, arrhythmia, measurement error, or other reasons. A patient monitoring system may receive one or more physiological signals, such as a photoplethysmograph (PPG) signal generated by a pulse oximeter sensor coupled to a patient. The patient monitoring system may generate a plurality of morphology metric signals from the PPG signal. In the absence of a gap in the data, an autocorrelation sequence may be generated for each of the morphology metric signals, the autocorrelation sequences may be combined, and the physiological information may be determined based on the combined autocorrelation sequence.

If there are gaps in the data it may not be desirable to utilize the portions of the signal corresponding to gaps to generate the autocorrelation sequences. The autocorrelation sequences may be used to identify periodic characteristics of the underlying morphology metric signals, and any gap portions of the data may skew the result. Removing the gap portion is not ideal, because the phase relationship between the remaining portions of the morphology metric signals may be lost, resulting in an inaccurate determination of the periodic characteristics of the morphology metric signals. Ignoring all data prior to and including the final gap portion of the signal may retain the phase relationship of the remaining portion of the signal, but reduces the amount of data available to determine the physiological information.

In an embodiment one or more gap portions may be identified for an analysis window of the physiological signal. The relative location of the gap portions and other information related to the gap portions may be stored. A plurality of morphology metric signals may be generated from the physiological signal, and the each of the morphology metric signals may be modified based on location of the gap portions and the gap information. An autocorrelation sequence may be generated for each of the modified morphology metric signals. A combined autocorrelation sequence may be generated from the autocorrelation sequences and the physiological information may be determined based on the combined autocorrelation sequence.

For purposes of clarity, the present disclosure is written in the context of the physiological signal being a PPG signal generated by a pulse oximetry system. It will be understood that any other suitable physiological signal or any other suitable system may be used in accordance with the teachings of the present disclosure.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I₀(λ)exp(−(sβ₀(λ)+(1−s)βᵣ(λ))/(t)) (1)

where:
λ=wavelength;
t=time;
I=intensity of light detected;
I₀=intensity of light transmitted;
s=oxygen saturation;
β₀,βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log Iₒ−(sβₒ+(1−s)βᵣ)1. (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{R}, in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for S yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that logA−logB=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

Where R represents the “ratio of ratios.”
8. Solving Eq. 4 for S using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{\frac{d I}{d t}}{I} , \qquad ( 10 )$$

Eq. 8 becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂,λ_{IR})−I(t₁,λ_{IR})]I(t₁,λ_{R}) (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

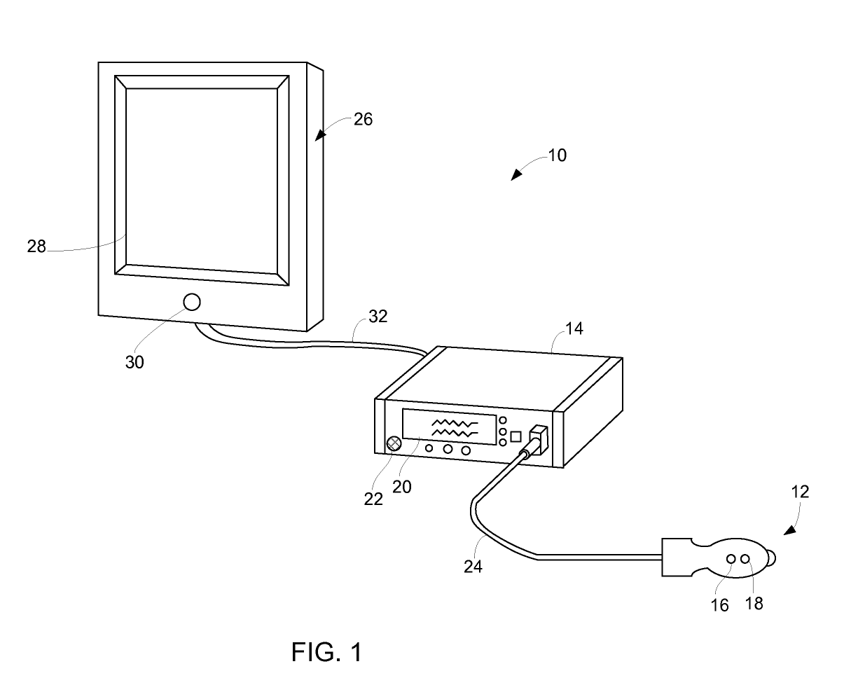

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to some embodiments, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood oxygen saturation, and respiration information) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may allow communication between monitor 14 and sensor unit 12.

Patient monitoring system 10 may also include display monitor 26. Monitor 14 may be in communication with display monitor 26. Display monitor 26 may be any electronic device that is capable of communicating with monitor 14 and calculating and/or displaying physiological parameters, e.g., a general purpose computer, tablet computer, smart phone, or an application-specific device. Display monitor 26 may include a display 28 and user interface 30. Display 28 may include touch screen functionality to allow a user to interface with display monitor 26 by touching display 28 and utilizing motions. User interface 30 may be any interface that allows a user to interact with display monitor 26, e.g., a keyboard, one or more buttons, a camera, a microphone, or a touchpad.

Monitor 14 and display monitor 26 may communicate utilizing any suitable transmission medium, including wireless (e.g., WiFi, Bluetooth, etc.), wired (e.g., USB, Ethernet, etc.), or application-specific connections. In an exemplary embodiment, monitor 14 and display monitor 26 may be connected via cable 32. Monitor 14 and display monitor 26 may communicate utilizing standard or proprietary communications protocols, such as the Standard Host Interface Protocol (SHIP) developed and used by Covidien of Mansfield, Mass. In addition, monitor 14, display monitor 26, or both may be coupled to a network to enable the sharing of information with servers or other workstations (not shown). Monitor 14, display monitor 26, or both may be powered by a battery (not shown) or by a conventional power source such as a wall outlet.

Monitor 14 may transmit calculated physiological parameters (e.g., pulse rate, blood oxygen saturation, and respiration information) to display monitor 26. In some embodiments, monitor 14 may transmit a PPG signal, data representing a PPG signal, or both to display monitor 26, such that some or all calculated physiological parameters (e.g., pulse rate, blood oxygen saturation, and respiration information) may be calculated at display monitor 26. In an exemplary embodiment, monitor 14 may calculate pulse rate and blood oxygen saturation, while display monitor 26 may calculate respiration information such as a respiration rate.

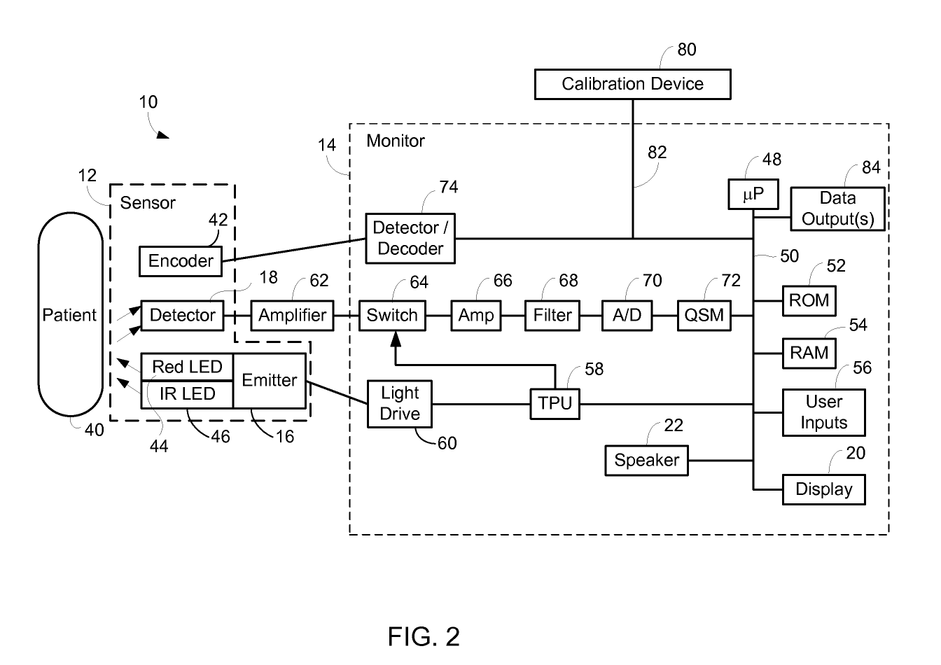

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor may emit only a Red light while a second sensor may emit only an IR light. In a further example, the wavelengths of light used may be selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which measurements may be determined based at least in part on the signal or signals received at sensor unit 12. For example, some pulse oximetry sensors rely on equations to relate an area under a portion of a PPG signal corresponding to a physiological pulse to determine blood pressure. These equations may contain coefficients that depend upon a patient's physiological characteristics as stored in encoder 42. Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics. In some embodiments, encoder 42 may include a memory on which one or more of the following information may be stored for communication to monitor 14; the type of the sensor unit 12; the wavelengths of light emitted by emitter 16; the particular wavelength each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, data output 84, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment.”

In some embodiments, microprocessor 48 may determine the patient's physiological parameters, such as Sp0₂, pulse rate, and/or respiration information, using various algorithms and/or look-up tables based on the value of the received signals and/or data corresponding to the light received by detector 18.

Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. In some embodiments, user inputs 56 may be used to enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

Data output 84 may provide for communications with other devices such as display monitor 26 utilizing any suitable transmission medium, including wireless (e.g., WiFi, Bluetooth, etc.), wired (e.g., USB, Ethernet, etc.), or application-specific connections. Data output 84 may receive messages to be transmitted from microprocessor 48 via bus 50. Exemplary messages to be sent in an embodiment described herein may include PPG signals to be transmitted to display monitor module 26.

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

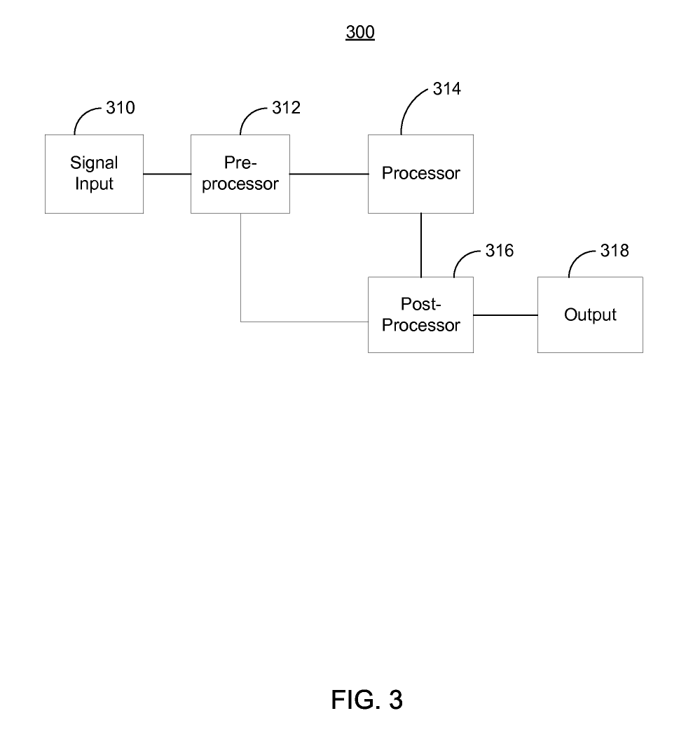

FIG. 3 is an illustrative processing system 300 in accordance with an embodiment that may implement the signal processing techniques described herein. In some embodiments, processing system 300 may be included in a patient monitoring system (e.g., patient monitoring system 10 of FIGS. 1-2). Processing system 300 may include input signal 310, pre-processor 312, processor 314, post-processor 316, and output 318. Pre-processor 312, processor 314, and post-processor 316 may be any suitable software, firmware, hardware, or combination thereof for calculating physiological parameters such as respiration information based on input signal 310. For example, pre-processor 312, processor 314, and post-processor 316 may include one or more hardware processors (e.g., integrated circuits), one or more software modules, computer-readable media such as memory, firmware, or any combination thereof. Pre-processor 312, processor 314, and post-processor 316 may, for example, be a computer or may be one or more chips (i.e., integrated circuits). Pre-processor 312, processor 314, and post-processor 316 may, for example, include an assembly of analog electronic components.

In some embodiments, processing system 300 may be included in monitor 14 and/or display monitor 26 of a patient monitoring system (e.g., patient monitoring system 10 of FIGS. 1-2). In the illustrated embodiment, input signal 310 may be a PPG signal. Input signal 310 may be a PPG signal that was sampled and generated at monitor 14, for example at 76 Hz. Input signal 310, pre-processor 312, processor 314, and post-processor 316 may reside entirely within a single device (e.g., monitor 14 or display monitor 26) or may reside in multiple devices (e.g., monitor 14 and display monitor 26).

Input signal 310 may be coupled to pre-processor 312. In some embodiments, input signal 310 may include PPG signals corresponding to one or more light frequencies, such as a Red PPG signal and an IR PPG signal. In some embodiments, the signal may include signals measured at one or more sites on a patient's body, for example, a patient's finger, toe, ear, arm, or any other body site. In some embodiments, signal 310 may include multiple types of signals (e.g., one or more of an ECG signal, an EEG signal, an acoustic signal, an optical signal, a signal representing a blood pressure, and a signal representing a heart rate). The signal may be any suitable biosignal or signals, such as, for example, electrocardiogram, electroencephalogram, electrogastrogram, electromyogram, heart rate signals, pathological sounds, ultrasound, or any other suitable biosignal. The systems and techniques described herein are also applicable to any dynamic signals, non-destructive testing signals, condition monitoring signals, fluid signals, geophysical signals, astronomical signals, electrical signals, financial signals including financial indices, sound and speech signals, chemical signals, meteorological signals including climate signals, any other suitable signal, and/or any combination thereof.

Pre-processor 312 may be implemented by any suitable combination of hardware and software. In an embodiment, pre-processor 312 may be any suitable signal processing device and the signal received from input signal 310 may include one or more PPG signals. An exemplary received PPG signal may be received in a streaming fashion, or may be received on a periodic basis as a sampling window, e.g., every 5 seconds. The received signal may include the PPG signal as well as other information related to the PPG signal, e.g., a pulse found indicator, the mean pulse rate from the PPG signal, the most recent pulse rate, an indicator of invalid samples, and an indicator of artifacts within the PPG signal. It will be understood that input signal 310 may include any suitable signal source, signal generating data, signal generating equipment, or any combination thereof to be provided to pre-processor 312. The signal received at input signal 310 may be a single signal, or may be multiple signals transmitted over a single pathway or multiple pathways.

Pre-processor 312 may apply one or more signal processing operations to input signal 310. For example, pre-processor 312 may apply a pre-determined set of processing operations to input signal 310 to produce a signal that may be appropriately analyzed and interpreted by processor 314, post-processor 316, or both. Pre-processor 312 may perform any necessary operations to provide a signal that may be used as an input for processor 314 and post-processor 316 to determine physiological information such as respiration information. Examples include reshaping the signal for transmission, multiplexing the signal, modulating the signal onto carrier signals, compressing the signal, encoding the signal, filtering the signal, low-pass filtering, band-pass filtering, signal interpolation, downsampling of a signal, attenuating the signal, adaptive filtering, closed-loop filtering, any other suitable filtering, and/or any combination thereof.

Other signal processing operations may be performed by pre-processor 312 for each pulse and may be related to producing morphology metrics suitable as inputs to determine physiological information. Pre-processor 312 may perform calculations based on an analysis window of a series of recently received PPG signal sampling windows, e.g., a 45-second analysis window may correspond to the 9 most recent 5-second sampling windows. The physiological information may be respiration information, which may include any information relating to respiration, e.g., respiration rate, change in respiration rate, breathing intensity, etc. Because respiration has an impact on pulse characteristics, it may be possible to determine respiration information from a PPG signal. Morphology metrics may be parameters that may be calculated from the PPG signal that provide information related to respiration. Examples include a down metric for a pulse, kurtosis for a pulse, the delta of the second derivative between predetermined samples of consecutive pulses, the up metric for a pulse, skew, ratio of predetermined samples of a pulse or its first or second derivative (e.g., b/a ratio or c/a ratio), peak amplitude of a pulse, center of gravity of a pulse, or area of a pulse, as described in more detail herein. Other information that may be determined by pre-processor 312 may include the pulse rate, the variability of the period of the PPG signal, the variability of the amplitude of the PPG signal, and an age measurement indicative of the age of the useful portion of the analyzed PPG signal.

In some embodiments, pre-processor 312 may be coupled to processor 314 and post-processor 316. Processor 314 and post-processor 316 may be implemented by any suitable combination of hardware and software. Processor 314 may receive physiological information and calculated parameters from pre-processor 312. For example, processor 314 may receive morphology metrics for use in calculating morphology metric signals that may be used to determine respiration information, as well as pulse rate and an age for the morphology metric signals. For example, processor 314 may receive samples representing a number of morphology metric values, such as down metric calculations, kurtosis metric calculations, delta of the second derivative (DSD) metric calculations, and b/a ratio calculations from pre-processor 312. The down metric is the difference between a first (e.g., fiducial) sample of a fiducial-defined portion of the PPG signal and a minimum sample of the fiducial-defined portion of the PPG signal. The DSD metric is the delta (difference) between fiducial points in consecutive fiducial-defined portions of the second derivative of the PPG signal.

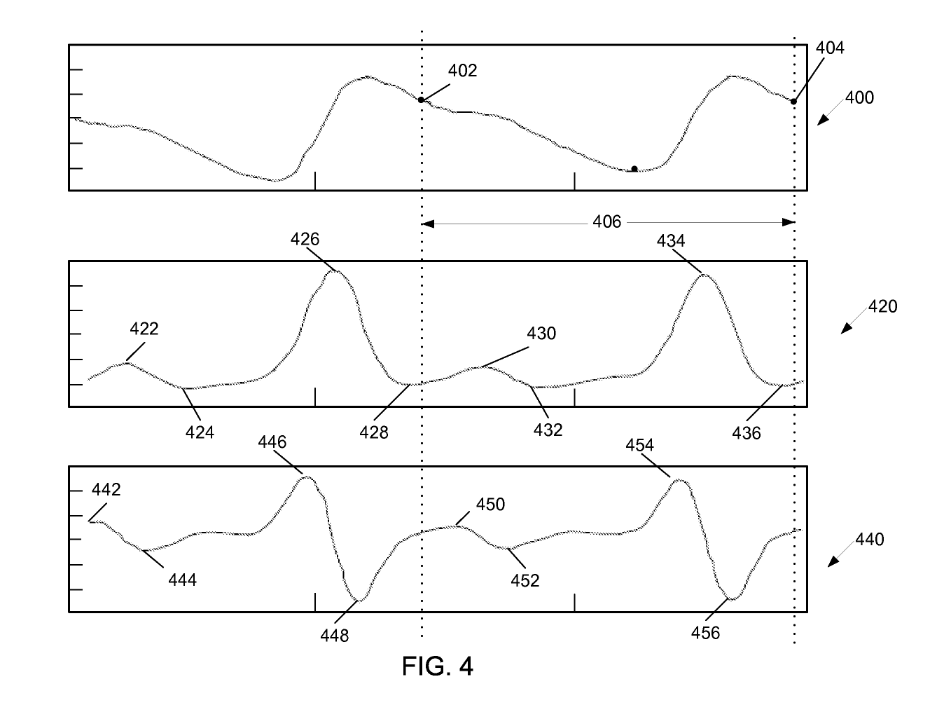

The b/a ratio metric (i.e., b/a) is depicted in FIG. 4. FIG. 4 depicts an exemplary PPG signal 400, first derivative of the PPG signal 420, and second derivative of the PPG signal 440. The b/a ratio is based on the ratio between the a-peak and b-peak of the PPG signal 400, first derivative signal 420, or second derivative signal 440. In FIG. 4, the b/a ratio is depicted for the first derivative signal 420 and second derivative signal 440. Fiducial points 402 and 404 of PPG signal 400 define a fiducial-defined portion 406. Each of PPG signal 400, first derivate signal 420, and second derivative signal 440 may include a number of peaks (e.g., four peaks corresponding to maxima and minima) which may be described as the a-peak, b-peak, c-peak, and d-peak, with the a-peak and c-peak generally corresponding to local maxima within a fiducial-defined portion and the b-peak and d-peak generally corresponding to local minima within a fiducial-defined portion. For example, for first derivative signal 420 the a-peaks are indicated by points 426 and 434, the b-peaks by points 428 and 436, the c-peaks by points 422 and 430, and the d-peaks by points 424 and 432. The b/a ratio measures the ratio of the b-peak (e.g., 428 or 436) and the a-peak (e.g., 426 or 434). For second derivative signal 440 the a-peaks are indicated by points 446 and 454, the b-peaks by points 448 and 456, the c-peaks by points 442 and 450, and the d-peaks by points 444 and 452. The b/a ratio measures the ratio of the b-peak (e.g., 448 or 456) and the a-peak (e.g., 446 or 454).

Kurtosis measures the peakedness of a signal, such as the PPG signal, the first derivative of the PPG signal, or the second derivative of the PPG signal. In an exemplary embodiment, the kurtosis metric may be based on the first derivative of the PPG signal. The kurtosis of a signal may be calculated based on the following formulae:

$$\begin{matrix}D = \frac{1}{n} {\sum }_{i = 1}^{n} {( {x}_{i}^{′} - {\overline{x}}^{′} )}^{2} & ( 14 ) \\ Kurtosis = \frac{1}{{nD}^{2}} {\sum }_{i = 1}^{n} {( {x}_{i}^{′} - {\overline{x}}^{′} )}^{4} & ( 15 )\end{matrix}$$

where:
xᵢ′=ith sample of 1^{st} derivative;
x̅′̅=mean of 1st derivative of fiducial-defined portion;
n=set of all samples in the fiducial-defined portion.

Processor 314 may utilize the received morphology metric values to calculate morphology metric signals and then to calculate respiration information signals and values from the morphology metric signals. Processor 314 may be coupled to post-processor 316 and may communicate respiration information to post-processor 316. Processor 314 may also provide other information to post-processor 316 such as the signal age related to the signal used to calculate the respiration information, a time ratio representative of the useful portion of the respiration information signal, or a confidence metric indicative of the strength of the respiration information signals. Pre-processor 312 may also provide information to post-processor 316 such as period variability, amplitude variability, and pulse rate information. Post-processor 316 may utilize the received information to calculate an output respiration information, as well as other information such as the age of the respiration information and status information relating to the respiration information output, e.g., whether a valid output respiration information value is currently available. Post-processor 316 may provide the output information to output 318.

Output 318 may be any suitable output device such as one or more medical devices (e.g., a medical monitor that displays various physiological parameters, a medical alarm, or any other suitable medical device that either displays physiological parameters or uses the output of post-processor 316 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

In some embodiments, all or some of pre-processor 312, processor 314, and/or post-processor 316 may be referred to collectively as processing equipment. For example, processing equipment may be configured to amplify, filter, sample and digitize an input signal 310 and calculate physiological information from the signal.

Pre-processor 312, processor 314, and post-processor 316 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by pre-processor 312, processor 314, and post-processor 316 to, for example, store data relating to input PPG signals, morphology metrics, respiration information, or other information corresponding to physiological monitoring.

It will be understood that system 300 may be incorporated into system 10 (FIGS. 1 and 2) in which, for example, input signal 310 may be generated by sensor unit 12 (FIGS. 1 and 2) and monitor 14 (FIGS. 1 and 2). Pre-processor 312, processor 314, and post-processor 316 may each be located in one of monitor 14 or display monitor 26 (or other devices), and may be split among multiple devices such as monitor 14 or display monitor 26. In some embodiments, portions of system 300 may be configured to be portable. For example, all or part of system 300 may be embedded in a small, compact object carried with or attached to the patient (e.g., a watch, other piece of jewelry, or a smart phone). In some embodiments, a wireless transceiver (not shown) may also be included in system 300 to enable wireless communication with other components of system 10 (FIGS. 1 and 2). As such, system 10 (FIGS. 1 and 2) may be part of a fully portable and continuous patient monitoring solution. In some embodiments, a wireless transceiver (not shown) may also be included in system 300 to enable wireless communication with other components of system 10. For example, communications between one or more of pre-processor 312, processor 314, and post-processor 316 may be over BLUETOOTH, 802.11, WiFi, WiMax, cable, satellite, Infrared, or any other suitable transmission scheme. In some embodiments, a wireless transmission scheme may be used between any communicating components of system 300.

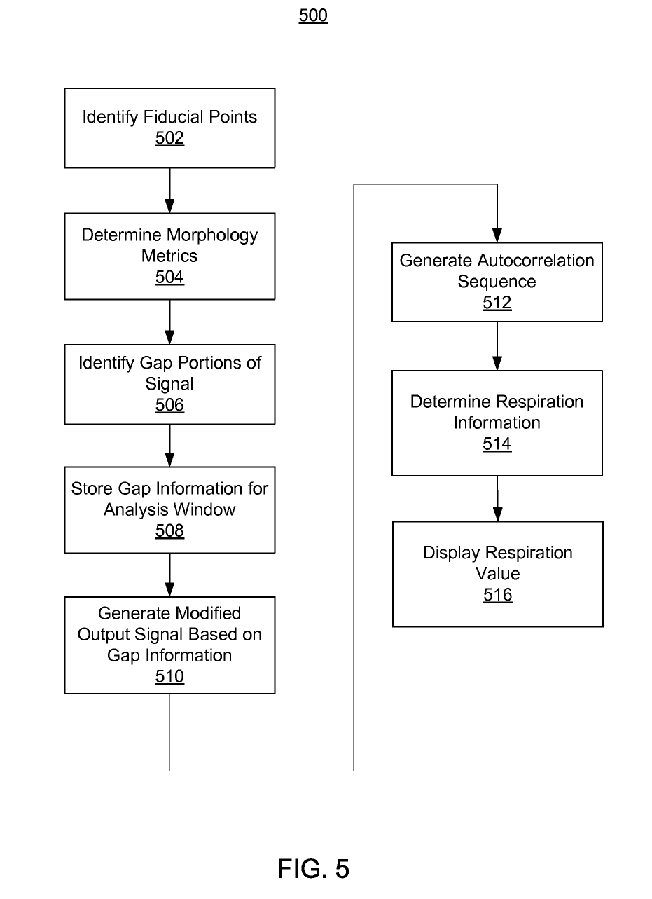

FIG. 5 depicts a flow diagram showing illustrative steps for determining a physiological parameter such as respiration information from a physiological signal such as a PPG signal in accordance with some embodiments of the present disclosure. Although an exemplary embodiment is described herein, it will be understood that each of steps 500 may be performed by pre-processor 312, processor 314, post-processor 316, or any combination thereof. It will also be understood that steps 500 may be performed in alternative sequence or in parallel, that steps may be omitted, and that additional steps may be added or inserted.

At step 502 pre-processor 312 may identify fiducial points for successive pulse waves of a PPG signal. Fiducial points may be identified in any suitable manner, for example based on peaks, troughs, slope values (e.g., the maximum slope of the PPG signal), and/or predetermined offsets. An example of determining fiducial points for a PPG signal is described in more detail in co-pending, commonly assigned U.S. patent application Ser. No. 13/243,907, filed Sep. 23, 2011 and entitled “SYSTEMS AND METHODS FOR DETERMINING RESPIRATION INFORMATION FROM A PHOTOPLETHYSMOGRAPH,” which is incorporated by reference herein in its entirety. The fiducial points may define a series of fiducial-defined portions that may be used as a reference for subsequent calculations, e.g., of morphology metric signals.

At step 504 pre-processor 312 may generate morphology metrics from the PPG signal. Morphology metrics may be calculated from the PPG signal in any suitable manner. In one embodiment, a plurality of morphology metrics may be generated from the PPG signal. Example morphology metrics that may be relevant to determining a physiological parameter such as respiration information from a PPG signal may include a down metric, a kurtosis metric, a delta of second derivative (DSD) metric, an up metric, a skew metric, a ratio of samples metric (e.g., a b/a ratio metric or c/a ratio metric), a i b metric, a peak amplitude metric, a center of gravity metric, and an area metric. In an exemplary embodiment, morphology metric signals may be generated for the down metric, kurtosis metric, DSD metric, and b/a ratio metric. For each morphology metric a set of morphology metric values, each corresponding to a fiducial defined portion, may be calculated. The sets of morphology metric values may be communicated to processor 314 to be attenuated, interpolated, and filtered to generate the morphology metric signals. Generating morphology metric signals from a PPG signal is described in more detail in co-pending, commonly assigned U.S. patent application Ser. No. 13/243,853, filed Sep. 23, 2011 and entitled “SYSTEMS AND METHODS FOR DETERMINING RESPIRATION INFORMATION FROM A PHOTOPLETHYSMOGRAPH,” which is incorporated by reference herein in its entirety.

At step 506, pre-processor 312, processor 314, or both, may identify one or more gap portions based on a signal. The gap portions may be identified based on any suitable signal and may be identified in any suitable manner, for example, based on portions of a signal that are likely to be indicative of a measurement error or physiological phenomena that may interfere with the calculation of physiological information. In an exemplary embodiment, pre-processor 312 may identify one or more gaps in the PPG signal, e.g., based on information relating to the input signal 310 such as indicators of the presence of a motion artifact or an indicator of invalid samples in the received PPG signal. Pre-processor 312 or processor 314 may also analyze the PPG signal to identify gaps, e.g., based on a large baseline shift identified in the PPG signal, an out of range pulse rate, or large variations in the pulse period. In some embodiments, pre-processor 312 or processor 314 may analyze the sets of morphology metric values or the morphology metric signals to identify gap portions, e.g., based on identifying an arrhythmia or an undesirable variation for the morphology metric.

At step 508, pre-processor 312, processor 314, or both, may store information related to the identified gap portions in any suitable manner. The physiological information may be determined by generating a plurality of autocorrelation sequences from the morphology metric signals, generating a combined autocorrelation sequence, and determining the physiological information from the combined autocorrelation sequence as described herein. Completely removing any gap portions from the morphology metric signals or the autocorrelation procedure and concatenating the remaining signal segments may result in the loss of the phase relationship between the remaining portions of the morphology metric signals, resulting in an inaccurate determination of the periodic characteristics of the morphology metric signals. Ignoring all data prior to and including the final gap portion of the signal may retain the phase relationship of the remaining portion of the signal, but reduces the amount of data available to determine the physiological information.

The phase relationship for the analysis window may be maintained despite the presence of gaps, e.g., by storing information relating to the relative position of any gaps within the analysis window. Position information relating to the gaps may be identified in any suitable manner. In an exemplary embodiment, gaps may be identified based on a continuous portion of the analysis window, i.e., for multiple samples within an analysis window corresponding to a gap. In some embodiments, the position of gaps within the analysis window may be identified on a sample-by-sample basis, or in the case of morphology metric values, for each fiducial-defined portion. In some embodiments, the position of gaps within the analysis window may be identified as any continuous portion of the analysis window having more than one gap within a predetermined interval, such as 5 seconds for a 45 second analysis window. The identified gaps may be stored in any suitable manner, such as in any suitable memory device associated with pre-processor 312 or processor 314. Additional information relating to the identified gaps may also be stored, such as the manner in which the gap was identified (e.g., artifact, out of range pulse rate, etc.) or a measure of the severity of the condition that resulted in the gap (e.g., a value from 0-1 indicating the severity of an arrhythmia condition, the variation of the pulse period, etc.).

At step 510, pre-processor 312, processor 314, or both, may generate a modified output signal based on the gap information. A modified output signal may be generated in any suitable manner. In an exemplary embodiment, a modified output signal may be a modified morphology metric signal. A plurality of sets of morphology metric values may be determined from the PPG signal and a plurality of morphology metric signals may be generated from the plurality of sets of morphology metric values, as described herein. A portion of each morphology metric signal may then be modified based the stored gap information. In an embodiment, any portion of each morphology metric signal corresponding to a gap portion may be modified. In an embodiment, any portion of a morphology metric signal corresponding to a gap may be set to zero. It will be understood that the portion of the morphology metric signal may be modified in any other suitable manner, such as attenuating the signal based on a measure of the severity of the condition that resulted in a gap. Other exemplary modifications of a signal include replacing the gap portions of the morphology metric signal with noise, generating substitute data for any gap portions of the morphology metric signal based on adjoining portions of the morphology metric signal, or generating substitute data having similar morphology, frequency, or amplitude characteristics to the remainder of the signal. It will be understood that while an exemplary embodiment has described modifying the morphology metric signals, other signals such as the PPG signal or the sets of morphology metric values may be modified in any suitable manner as described herein. In these embodiments the PPG signal or sets of morphology metric values may be modified, and the morphology metric signals may be generated based on the modified PPG signal or sets of morphology metric signals.

In some embodiments, step 510 may not be performed, i.e., the morphology metric signals (or the PPG signal or sets of morphology metric values) may be generated without modifying the signals based on gaps. In an embodiment, other steps such as generating autocorrelation sequences may be adjusted based on the gap portions. The calculation of each value in the autocorrelation sequence may be modified such that any portion of the correlation corresponding to a gap is ignored or otherwise reduced.

Although steps 506, 508, and 510 have been described with respect to a particular embodiment, it will be understood that the steps may be modified in any suitable manner to identify gaps and modify one or more signals based on the gaps. For example, different morphology metric signals may relate to different phenomena that are indicative of respiration, and gaps may be identified based on signal characteristics that relate differently to each of the morphology metric signals. In an embodiment, one or more morphology metric signals may be modified in a different manner based on the type of gap that is identified. It will also be understood that the steps of identifying gap portions (step 506) and storing gap information for the analysis window (step 508) may be performed at any suitable time. In an exemplary embodiment gap portions may be identified from a PPG signal and the signal to be modified may be a morphology metric signal. The gap portions may be identified and stored at any time before the morphology metric signals are used to determine respiration information, e.g., at any time before an autocorrelation sequence is generated for each of the morphology metric signals as described herein.

Processing may continue at step 512. At step 512, processor 314 may generate an autocorrelation sequence for each morphology metric signal. Although an autocorrelation sequence may be generated in any suitable manner, in one embodiment an autocorrelation sequence may be generated for each morphology metric signal and the autocorrelation sequences may be combined into a single autocorrelation sequence based on weighting factors. Generating the autocorrelation sequence from the morphology metric signals is described in more detail in co-pending, commonly assigned U.S. patent application Ser. No. 13/243,951, filed Sep. 23, 2011 and entitled “SYSTEMS AND METHODS FOR DETERMINING RESPIRATION INFORMATION FROM A PHOTOPLETHYSMOGRAPH,” which is incorporated by reference herein in its entirety.

As described herein, one or more of the morphology metric signals may be modified based on one or more identified gaps (or may be based on a PPG signal or set of morphology metric values that was modified). Each autocorrelation sequence may be generated in the normal manner based on the modified morphology metric signals. In some embodiments the morphology metric signals need not be modified based on identified gap portions. In this embodiment the procedure for generating the autocorrelation sequence may be modified based on the stored gap information. Although generating the autocorrelation sequence may be modified in any suitable manner, in an exemplary embodiment any portion of the morphology metric signal corresponding to a gap portion may be ignored or reduced in calculating each value in the autocorrelation sequence. For each autocorrelation value in the autocorrelation sequence, portions of the correlation having any overlap with a gap portion may be reduced (e.g., by a scaling factor) or set to zero. It will be understood that zeroing in this way (i.e., zeroing portions the underlying morphology metric signals or zeroing a window of data during the generation of the autocorrelation sequences) is similar to applying rectangular windows to the signal. As in other branches of signal processing, in certain circumstances it may prove beneficial to apply a different shaped window to the data (e.g. Hann, Hamming, Cosine, etc.) to optimize the autocorrelation output.

At step 514, processor 314 may determine respiration information based on the combined autocorrelation sequence. Respiration information may be determined from the autocorrelation sequence in any suitable manner. In one exemplary embodiment, a continuous wavelet transform may be used to determine respiration information such as respiration rate from the autocorrelation sequence, as is described in more detail in co-pending, commonly assigned U.S. patent application Ser. No. 13/243,892, filed Sep. 23, 2011 and entitled “SYSTEMS AND METHODS FOR DETERMINING RESPIRATION INFORMATION FROM A PHOTOPLETHYSMOGRAPH,” which is incorporated by reference herein in its entirety. In another exemplary embodiment, respiration information may be determined directly from the autocorrelation sequence, e.g., by comparing the peaks of the autocorrelation sequence to a threshold value or by identifying a maximum peak of the autocorrelation sequence within a window of interest. Determining respiration information directly from the autocorrelation sequence is described in more detail in co-pending, commonly assigned U.S. patent application Ser. No. 13/243,785, filed Sep. 23, 2011 and entitled “SYSTEMS AND METHODS FOR DETERMINING RESPIRATION INFORMATION FROM A PHOTOPLETHYSMOGRAPH,” which is incorporated by reference herein in its entirety. The output respiration value may be communicated to post-processor 316.

At step 516, post-processor 316 may determine a display respiration value to be displayed, e.g., at the patient monitoring system. The display respiration value may be determined in any suitable manner. For example, the display respiration value may be based on the currently received respiration value. In another exemplary embodiment, the display respiration value may be based on the received respiration value as well as previously received respiration values. In an exemplary embodiment, post-processor 316 may calculate the display respiration value from the respiration value for the current analysis window and respiration values for one or more previous analysis windows, e.g., the five previous analysis windows.

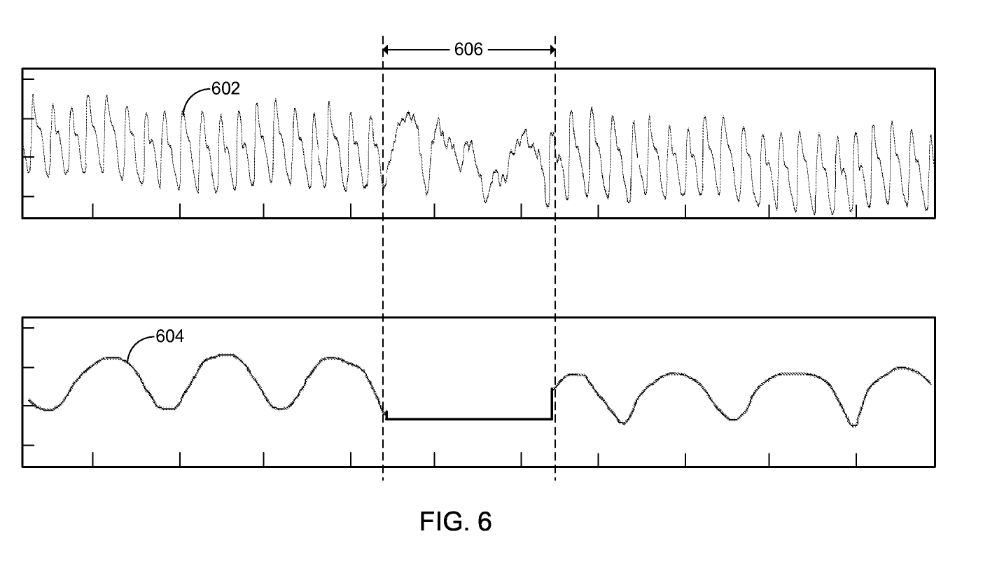

FIG. 6 depicts a plot of a physiological signal having a single gap portion and an exemplary morphology metric signal generated from the physiological signal. The abscissa of the plots of FIG. 6 may be in units of time and the ordinate in units of magnitude. The physiological signal may be a PPG signal 602, and may represent an analysis window of received data. Morphology metric signal 604 may be an exemplary morphology metric signal generated from PPG signal 602. PPG signal 602 may include a gap portion 606 having a portion of gap data, e.g., due to a motion artifact as described herein. In an exemplary embodiment, all portions of morphology metric signal 604 corresponding to the gap portion may be set to zero as depicted in FIG. 6.

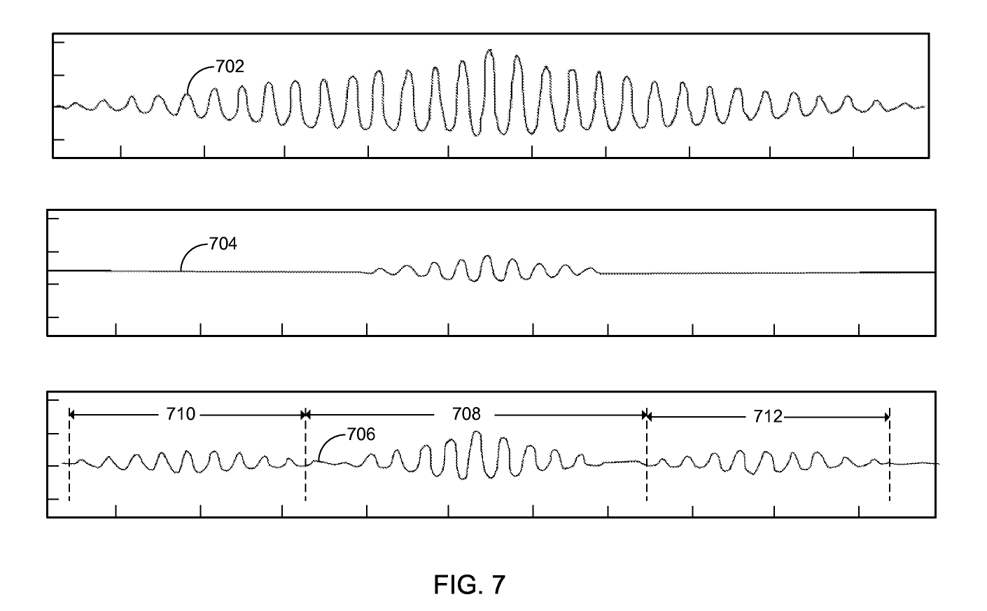

FIG. 7 depicts exemplary autocorrelation plots generated based on the physiological signal of FIG. 6. The abscissa of the plots of FIG. 7 may be in units of time and the ordinate in units of magnitude. Autocorrelation sequence 702 may be an exemplary result obtained from an underlying signal without gaps, e.g., a hypothetical PPG signal 602 wherein there is good data in place of gap portion 606. A center peak corresponds to the comparison of the signal with itself, and each additional peak indicates a periodic characteristic of the underlying signal, i.e., where the time shifted morphology metric signal is highly similar to itself. Autocorrelation sequence 704 may correspond to an autocorrelation sequence determined only from the portion of morphology metric signal 604 following gap portion 606. It may be desirable to maintain the proper phase relationship of morphology metric signal 604 within the analysis window in order to properly identify periodic characteristics of the signal. One option for maintaining the proper phase relationship for the signal is to ignore all portions of the morphology metric signal 604 prior to and including the gap portion 606. This reduces the amount of information available for calculating the autocorrelation sequence and therefore reduces the magnitude of the resulting autocorrelation sequence 704.

Autocorrelation sequence 706 may correspond to an autocorrelation sequence determined from all portions of the modified morphology metric signal 604. Autocorrelation sequence 706 may have a center region 708 and side lobes 710 and 712. Because autocorrelation sequence 706 is based on the complete modified morphology metric signal 604, significantly more information may be available for generating autocorrelation sequence 706. Center region 708 may exhibit higher peaks than autocorrelation sequence 704, resulting in a more accurate determination of periodic information such as respiration information. In an embodiment, autocorrelation signal 706 may tail off and then again increase in magnitude, resulting in side lobes 710 and 712. This feature may be characteristic of values of the autocorrelation sequence 706 wherein the gap (e.g., zeroed) portions of the modified morphology metric signal 604 correlate with the remaining (e.g., data) portions of the modified morphology metric signal 604. The side lobes 710 and 712 may be generated as the gap portion of the modified morphology metric signal 604 begins to fall out of the autocorrelation.

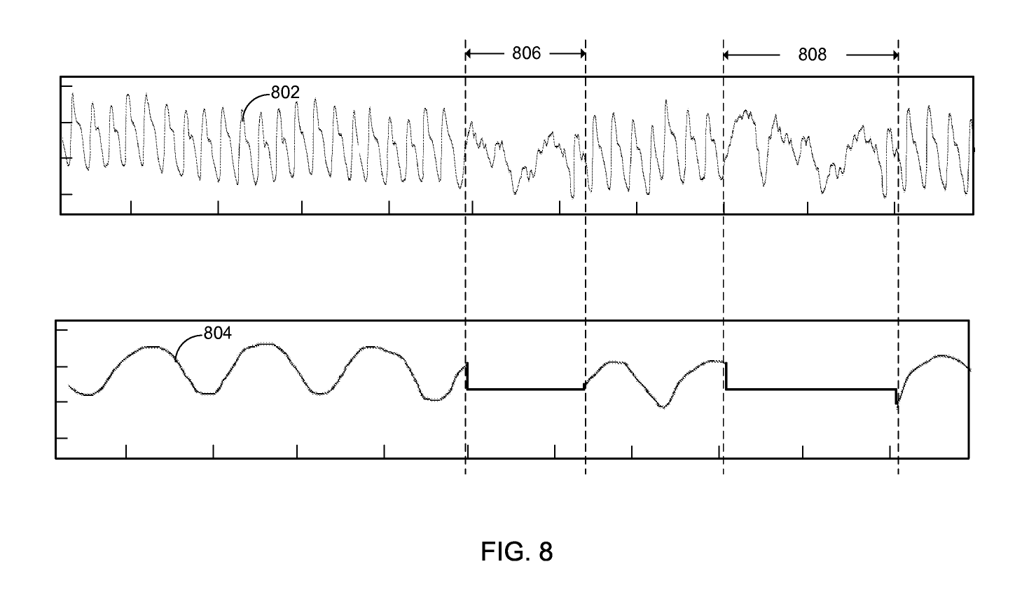

FIG. 8 depicts a plot of a physiological signal having two gap portions and an exemplary morphology metric signal generated from the physiological signal. The abscissa of the plots of FIG. 8 may be in units of time and the ordinate in units of magnitude. The physiological signal may be a PPG signal 802, and may represent an analysis window of received data. Morphology metric signal 804 may be an exemplary morphology metric signal generated from PPG signal 802. PPG signal 802 may include gap portions 806 and 808, each having a portion of gap data, e.g., due to a motion artifact as described herein. In an exemplary embodiment, all portions of morphology metric signal 804 corresponding to the gap may be set to zero as depicted in FIG. 8.

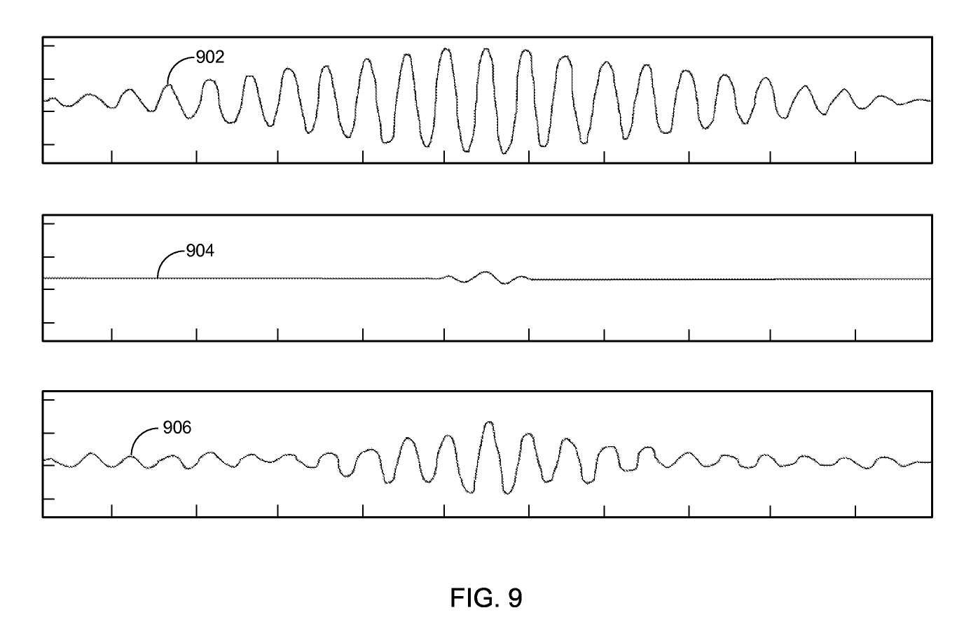

FIG. 9 depicts exemplary autocorrelation plots generated based on the physiological signal of FIG. 8. The abscissa of the plots of FIG. 9 may be in units of time and the ordinate in units of magnitude. Autocorrelation sequence 902 may be an exemplary result obtained from an underlying signal without gaps, e.g., a hypothetical PPG signal 802 wherein there is good data in place of gap portions 806 and 808. A center peak corresponds to the comparison of the signal with itself, and each additional peak indicates a periodic characteristic of the underlying signal, i.e., where the time shifted morphology metric signal is highly similar to itself. Autocorrelation sequence 904 may correspond to an autocorrelation sequence determined only from the portion of morphology metric signal 804 following gap portion 808. It may be desirable to maintain the proper phase relationship of morphology metric signal 804 within the analysis window in order to properly identify periodic characteristics of the signal. One option for maintaining the proper phase relationship for the signal is simply to ignore all portions of the morphology metric signal 804 prior to and including the final gap portion 808. This reduces the amount of information available for calculating the autocorrelation sequence and therefore reduces the magnitude of the resulting autocorrelation sequence 904.

Autocorrelation sequence 906 may correspond to an autocorrelation sequence determined from all portions of the modified morphology metric signal 804. Because autocorrelation sequence 906 is based on the complete modified morphology metric signal 804, significantly more information may be available for generating autocorrelation sequence 906. Autocorrelation sequence 906 may exhibit higher peaks than autocorrelation sequence 904, resulting in a more accurate determination of periodic information such as respiration information. In an embodiment, autocorrelation signal 906 may demonstrate varied peak heights rather than tailing off uniformly. This feature may be characteristic of values of the autocorrelation sequence 906 wherein the gap (e.g., zeroed) portions of the modified morphology metric signal 804 correlate with the remaining (e.g., data) portions of the modified morphology metric signal 804.

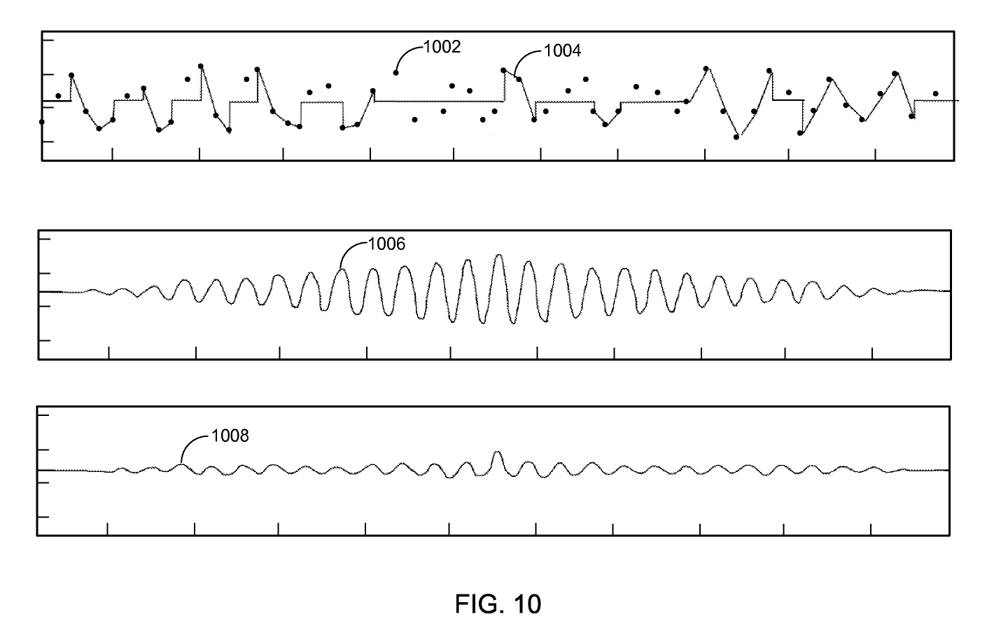

FIG. 10 depicts a plot of an exemplary physiological signal modified for fiducial-defined portions, and exemplary autocorrelation plots based on the physiological signal. The abscissa of the plots of FIG. 10 may be in units of time and the ordinate in units of magnitude. In an exemplary embodiment points 1002 may be morphology metric values determined from a PPG signal. Morphology metric signal 1004 may be a morphology metric signal constructed from a subset of the points 1002. Morphology metric signal 1004 may have portions that are modified (i.e., zeroed out in an exemplary embodiment) based on points 1004 that correspond to identified gaps. In the exemplary embodiment of FIG. 10, gaps may be identified and the signal may be modified on a sample-by-sample basis, i.e., for each morphology metric value corresponding to a gap the morphology metric signal may be modified. Autocorrelation sequence 1006 may be an exemplary result obtained from an underlying signal without gaps, e.g., a hypothetical morphology metric signal 1004 wherein there is good data in place of gap portions associated with points 1002. A center peak corresponds to the comparison of the signal with itself, and each additional peak indicates a periodic characteristic of the underlying signal, i.e., where the time shifted morphology metric signal is highly similar to itself. Autocorrelation sequence 1008 may correspond to an autocorrelation sequence determined from modified morphology metric signal 1004. Although there may be a limited amount of available information for generating an autocorrelation sequence, autocorrelation sequence 1008 may include sufficient data to determine periodic information such as respiration information.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A method for determining physiological information, the method comprising:
receiving a physiological signal;
identifying, using processing equipment, a gap portion for the physiological signal;
generating, using the processing equipment, a morphology metric signal based on the physiological signal;
modifying, using the processing equipment, the morphology metric signal based on the gap portion;
generating, using the processing equipment, an autocorrelation sequence based on the modified morphology metric signal; and
determining, using the processing equipment, the physiological information based on the autocorrelation sequence.

2. The method of claim 1, wherein modifying the morphology metric signal comprises attenuating a portion of the morphology metric signal that corresponds to the gap portion.

3. The method of claim 1, wherein modifying the morphology metric signal comprises zeroing a portion of the morphology metric signal that corresponds to the gap portion.

4. The method of claim 1, wherein modifying the morphology metric signal comprises replacing a portion of the morphology metric signal that corresponds to the gap portion with a noise signal.

5. The method of claim 1, wherein modifying the morphology metric signal comprises generating a replacement signal for the portion of the morphology metric signal that corresponds to the gap portion.

6. The method of claim 1, wherein identifying the gap portion comprises identifying a plurality of continuous portions of the physiological signal.

7. The method of claim 1, wherein identifying the gap portion comprises identifying a plurality of samples of the physiological signal.

8. The method of claim 1, further comprising:
generating, using the processing equipment, a plurality of morphology metric signals based on the physiological signal;
modifying, using the processing equipment, the plurality of morphology metric signals based on the gap portion;
generating, using the processing equipment, a combined autocorrelation sequence based on the plurality of modified morphology metric signals; and
determining, using the processing equipment, physiological information based on the combined autocorrelation sequence.

9. A method for determining physiological information, the method comprising:
receiving a physiological signal;
identifying, using processing equipment, a gap portion for the physiological signal;
generating, using the processing equipment, a morphology metric signal based on the physiological signal;
generating, using the processing equipment, a modified autocorrelation sequence based on the morphology metric signal and the gap portion; and
determining, using the processing equipment, physiological information based on the modified autocorrelation sequence.

10. The method of claim 9 wherein generating the modified autocorrelation sequence comprises modifying autocorrelation values based on the gap portion.

11. The method of claim 10 wherein modifying the autocorrelation values comprises ignoring any portion of the correlation that corresponds to the gap portion.

12. The method of claim 9, wherein identifying the gap portion comprises identifying a plurality of continuous portions of the physiological signal.

13. A patient monitoring system comprising:
an interface configured to receive a physiological signal; and
a processor configured to:
identify a gap portion for the physiological signal;
generate a morphology metric signal based on the physiological signal;
modify the morphology metric signal based on the gap portion;
generate an autocorrelation sequence based on the modified morphology metric signal; and
determine the physiological information based on the autocorrelation sequence.

14. The patient monitoring system of claim 13, wherein the processor is further configured to attenuate a portion of the morphology metric signal that corresponds to the gap portion.

15. The patient monitoring system of claim 13, wherein the processor is further configured to zero a portion of the morphology metric signal that corresponds to the gap portion.

16. The patient monitoring system of claim 13, wherein the processor is further configured to replace a portion of the morphology metric signal that corresponds to the gap portion with a noise signal.

17. The patient monitoring system of claim 13, wherein the processor is further configured to generate a replacement signal for the portion of morphology metric signal that corresponds to the gap portion.

18. The patient monitoring system of claim 13, wherein the processor is further configured to identify a plurality of continuous portions of the physiological signal.

19. The patient monitoring system of claim 13, wherein the processor is further configured to identify a plurality of samples of the physiological signal.

20. The patient monitoring system of claim 13, wherein the processor is further configured to:
generate a plurality of morphology metric signals based on the physiological signal;
modify the plurality of morphology metric signals based on the gap portion;
generate a combined autocorrelation sequence based on the plurality of modified morphology metric signals; and
determine the physiological information based on the combined autocorrelation sequence.
